(19) United States
(12) Patent Application Publication
(10) Pub. No.: US 2006/0285104 A1
(43) Pub. Date: Dec. 21, 2006

(54) Molecular imager

(76) Inventor: Meagan Walsh, Spokane Valley, WA (US)

Correspondence Address: NATIONAL IP RIGHTS CENTER, LLC;SCOTT J. FIELDS, ESQ., 550 TOWNSHIP LINE ROAD, SUITE 400, BLUE BELL, PA, 19422, US

(21) Appl. No.: 11/454,644

(22) Filed: Jun. 15, 2006

Related U.S. Application Data
(60) Provisional application No. 60/690,732, filed on Jun. 15, 2005.

(51) Int. Cl. G01N 33/48

(52) U.S. Cl. 356/39

(57) ABSTRACT

13 An apparatus for testing and determining a biological function based upon blood samples comprising a specimen holder for holding a blood sample; a light on the specimen; a molecular imager for viewing the specimens; and a software program for determining a medical problem based upon an aspect of the blood sample specimen.

### CROSS REFERENCE TO RELATED APPLICATIONS

This application claims benefit to provisional application 60/690,732, filed on Jun. 15, 2005 entitled Molecular Imager, which is incorporated herein in it's entirely.

### FIELD OF THE INVENTION

The present invention is directed to the field of blood cell imaging. In particular, the present invention is directed to an apparatus for in-home imaging and analysis of blood cells to identify a user's lack or excess of key enzymes, vitamins, and nutrients and to monitor an overall biochemical balance.

### BACKGROUND OF THE INVENTION

For years, pharmaceutical companies have attempted to dominate the burgeoning and lucrative health care market by offering drugs focused on treating the symptoms of illnesses. However, this focus has provided an untapped niche in preventative medicine and the highly effective tools to meet today's consumer's quest for healthy and full lives. Baby Boomers, now aged 35-58, do not “see” themselves as one of the seniors paying Four Thousand Seven Hundred Dollars ($4,700.00) a year for prescriptions. They also vividly imagine themselves as strong, healthy, and attractive at age ninety or even one hundred.

Currently one-third of the United States adult population is on some sort of diet or weight loss program. Every program promises the same thing; the loss of unwanted weight and the gain of a great body. The use of a molecular imager takes that promise a step further, it promises weight loss, but more importantly it promises to make the consumer medically fit and feel great. Unlike other programs, success is not measured by a weight scale; it is measured at the molecular level through the microscope imager. No other diet program or health care plan can offer what the Molecular Imager can.

The knowledge behind the Molecular Imager was founded in a large part due to two prominent and Nobel winning scientists, Frits Zernike and Linus Carl Pauling.

Zernike won the Nobel Prize in physics in 1953 for his work on phase contrast lenses, which makes it possible to observe microorganisms in a much greater detail than was ever before possible, and which now makes it feasible to view live blood cells. In 1954 Linus Carl Pauling was awarded a Nobel Prize in chemistry for his research into the orthomolecular nature of chemical bonds and their application to the elucidation of the structures of more complex substances.

The breakthroughs that Zernike and Pauling have accomplished are now being paired to introduce a new tool for the next century in home health care. Traditionally, the health market has been dominated by pharmaceutical companies which focus on medicating illnesses after they have occurred. The Imager reverses this trend and allows health conscientious customers to identify possible trouble areas before they appear.

In the trillion dollar wellness industry, the technological preventative methods have already inspired countless clients to change their lives in order to follow the advice given. With the inception of the Molecular Imager, countless more will be able to purchase a product that will benefit their lives far into the future.

There have been a number of patents directed to the area of blood imaging technologies. U.S. Pat. No. 5,934,278 discloses a non-invasive blood analyzer, having a light applying device for applying light to a detection region, including a blood vessel in a living body and a capturing device for capturing an image of the detection region to which the light is applied. Finally, an analyzing device is included for further processing the captured image to analyze blood cells in the blood vessel included in the detection region. Preferably, the analyzing device includes a reference image forming device for forming a reference image by using at least one of a plurality of images which the capturing device repeatedly captures with respect to the same detection region. In addition, it includes a differential image forming device for calculating a difference in pixel information between the reference image and one of the plurality of images to form a differential image by using the calculated difference as pixel information. Finally, a blood cell image detecting device is included for detecting a blood cell image from the differential image.

U.S. Pat. No. 6,746,401 discloses a system and method for visualizing ablation lesions. A contrast agent is injected into an artery that feeds blood to tissue surrounding an ablation lesion. During or after contrast agent injection, the ablation lesion and surrounding tissue is imaged using an imager that is responsive to the injected contrast agent. In one non-limiting embodiment, the contrast agent comprises echogenic particles, e.g., echogenic microbubbles, and the imager is an ultrasound imager.

U.S. Pat. No. 6,522,911 discloses a first measuring light beam of wavelength .lambda..sub.1 equal to a wavelength at the isosbestic point between oxyhemoglobin and deoxyhemoglobin and a second measuring light beam of wavelength .lambda..sub.2 differing from the first measuring light beam are incident on the same part of a subject such as a human finger and scan the subject by using an X-Y stage movable in X and Y directions. The first measuring light beam branches into two light beams. One of the two light beams is subjected to a frequency shift by a frequency shifter, while the other is transmitted through the subject. Thereafter, the two light beams are synthesized and a beat component of the synthesized first measuring light beam is detected by a first signal detection section. The first signal detection section outputs a first beat-component detection signal. For the second measuring light beam, a second beat-component detection signal is output in the same manner as the first measuring light beam. A personal computer generates an image signal based on a value of the second beat-component detection signal normalized by the first beat-component detection signal.

U.S. Pat. No. 6,171,246 discloses a perfusion imager. Perfusion of tissue such as the myocardium by a microbubble contrast agent is imaged in realtime by a multiple pulse technique which uses low power transmit pulses which are sufficient to elicit a harmonic response from the microbubbles but are not high enough in power to cause substantial destruction to the microbubbles. In a preferred embodiment the realtime microbubble image sequence is displayed in color, overlaid with a grayscale image of the surrounding tissue in the region being imaged.

U.S. Pat. No. 5,741,213 discloses an apparatus for analyzing blood which includes an image capturing device for capturing an image including at least one object blood cell and an analyzer for analyzing the captured image as an image F(x, y) in an x-y coordinate system. The analyzer includes an edge calculator for calculating an edge intensity distribution E(x, y) representing an outline of the image F(x, y); a weight memory for storing in advance a weight distribution W(i, j) corresponding to an average outline of the object cell; an assessment value calculator for obtaining an assessment value C (x, y) at each point (x, y) by calculating a degree of correspondence between the edge intensity distribution E(x, y) and the weight distribution W(i, j) for each point (x, y); and an extractor for extracting a point (x, y) at which the assessment value C(x, y) is larger than a predetermined value, thereby determining that the object blood cell is present at the point (x, y). The apparatus is useful as a device for identifying the object blood cell.

U.S. Pat. No. 4,998,533 discloses an apparatus and method for in vivo determination of red and white blood cell characteristics from a flow of red and white blood cells in mucous membranes are provided wherein image capturing means are employed to optically isolate images from a flow of blood cells and transmit those images to an image receiving means for encoding into electronic signals. The images are also filtered through light filters and subsequently transmitted to a computer for evaluation and analysis. The results of this analysis are then made available for display either on a CRT or hard copy printer.

U.S. Pat. No. 4,609,872 discloses a method for magnetic resonance imaging of fluid flow, and particularly in vivo blood flow, uses multiple-echo phase-contrast sequences of signals both in the magnetic field gradient in the direction in which fluid flow is to be determined, and in the radio-frequency (RF) magnetic field utilized with the magnetic field gradient. The magnetic field gradient has a pair of phase-encoding pulses having a zero mean value effect upon the sample, either alone or with inversion by an intermediate 180. degree. RF pulse signal. A first multi-echo sequence, provided with the phase-encoding pulse pair, provides information of both the amplitude and phase-shift of each pixel of the imaged flowing material while a second multiple echo sequence, devoid of the phase-encoding-gradient pulses, provides information as to the amplitude and the undesired initial phase-shift of each pixel of both stationary and flowing material. Information provided responsive to the second sequence is subtracted from the information provided responsive to the first sequence to provide an image having an intermediate grey-scale value for stationary sample material and with differential grey-scale encoding for differential flow velocities of fluid passing through the image plane.

Japanese Patent No. JP7,020,124 discloses a blood cell analyzer for realizing quick inspection, while saving manpower when the blood cells are classified. The blood cell analyzer comprises an automatic microscope 3 for magnifying a blood cell sprayed onto a slide glass 1a, a color TV camera 6 for picking up the microscopic image thereof, a section 9 for extracting the feature parameters of the blood cell from the image thus picked up, and an identifying/classifying section 10 for comparing the feature parameters of blood cell thus extracted with a preset feature parameter to classify and count the blood cell. The analyzer further comprises means for classifying the blood cells which could not be classified at the section 10 into one or more preset candidate types, means for counting the blood cells classified by the candidate blood cell type classifying means for each type, and means for deciding the necessity of review based on preset conditions for each slide class thus counted.

It is an object of the invention to provide a system for testing blood and identifying medical conditions using a molecular imager.

It is a further object of the invention to provide a system by which specific conditions can be determined.

It is a further object of the invention to provide a system which can be used as a home-based diagnostic tool.

These and other objects of the invention will become clear from the detailed description which follows.

### SUMMARY OF THE INVENTION

The present invention is directed to an apparatus which utilizes a Molecular Imager using the Frits Zernike method of phase contrast imaging, a high magnification lens, and an image capturing device to deliver an image of the user's blood cells to a computer screen or other viewing apparatus. Additionally, a computer program would be included that assists the user in identifying and interpreting the images displayed on the computer screen.

In accordance, the invention is an apparatus for testing and determining biological function based upon blood samples comprising of a specimen holder for holding a blood sample; a light on the specimen; a molecular imager for viewing the specimens; and a software program for determining a medical problem based upon an aspect of the blood sample specimen.

In a further embodiment, the invention is an apparatus for testing and determining a biological feature of a blood sample comprising of a specimen holder for holding a blood sample; a light for lighting the specimen of blood; a molecular imager for imaging the specimen; and a software program under the control of a microprocessor for determining a dietary problem based upon a feature of a blood sample specimen.

In still a further embodiment, the invention is an apparatus for testing and determining a biological feature of a blood sample comprising of a specimen holder for holding a blood sample; a light for lighting the specimen of blood; a molecular imager for imaging the specimen; a software program under the control of a microprocessor for determining a dietary problem based upon a characteristic of the blood sample specimen; and an output display for displaying the determined dietary problem.

### DESCRIPTION OF THE FIGURES

FIG. 1 is a side perspective view of the molecular imager of the present invention.

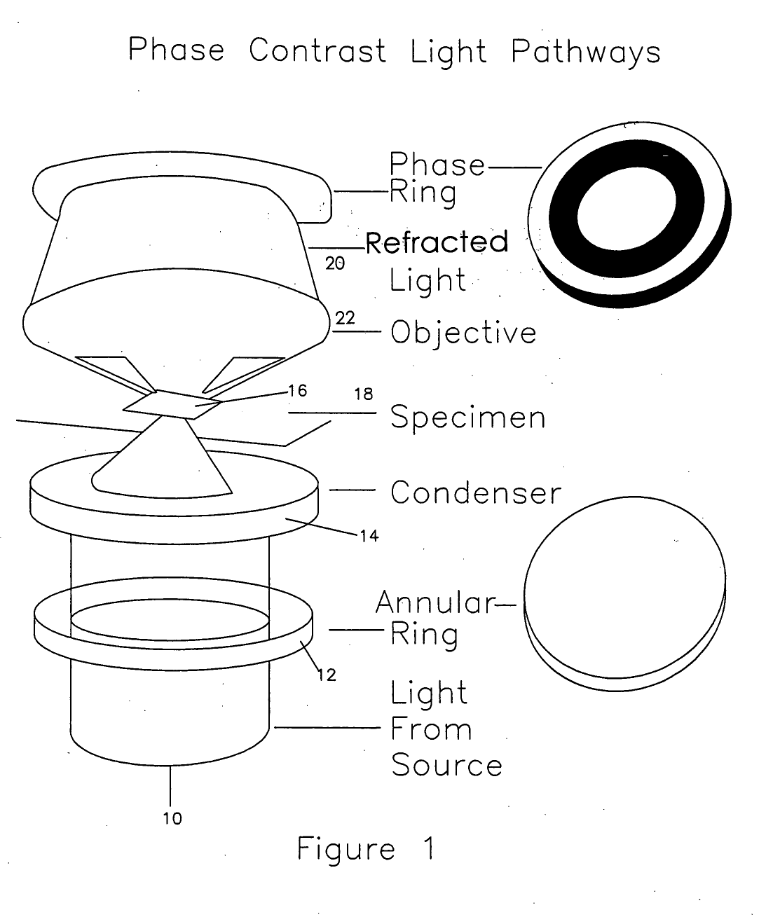

FIG. 2 is an overhead view of a home based imaging system in accordance with the present invention.

FIG. 3 is a block diagram of the control system of the present invention.

FIG. 4 is an elevational view of the present invention.

### DESCRIPTION OF THE PREFERRED EMBODIMENT

The present invention is a system for diagnosing and identifying vitamin, protein and mineral deficiencies in the body based upon an analysis of the blood and specifically utilizing a molecular image with software. In the preferred embodiment, the invention comprises a molecular imaging system 10. The system comprises a light source 10 which supports an annular ring 12 with condenser 14. A blood specimen 18 is placed on a specimen plate 16. Refracted light 20 is shown through an objective 22 on to the specimen plate 16.

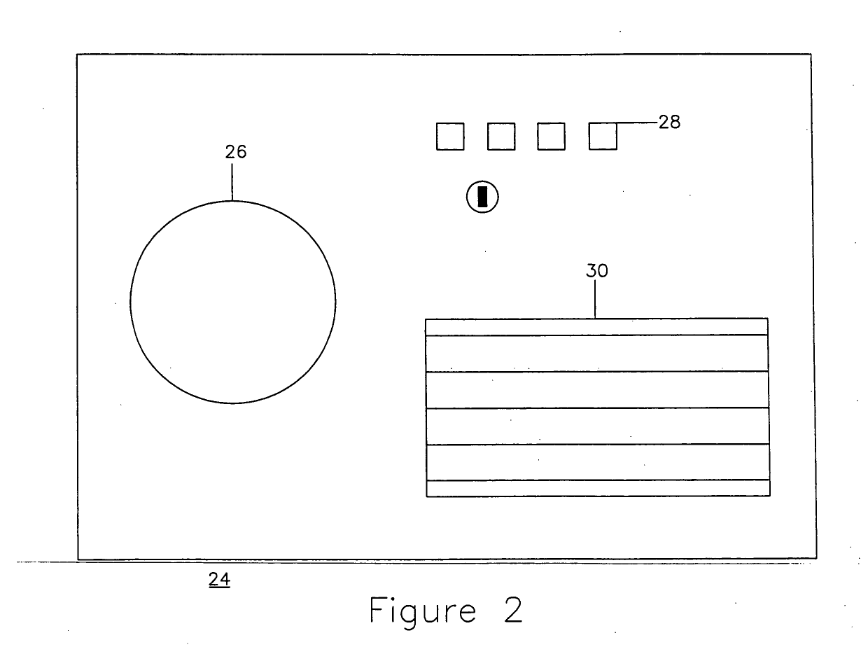

Referring to FIG. 2, the invention is configured to work with a home-based monitor diagnostic tool. The system and special software analyze the sample and provide diagnostic information based upon pre-stored algorithms. In one embodiment, the monitor comprises a module 24. The module 24 has a cylindrical aperture 26 for securing the molecular imager 10. Control buttons 28 control the operation of the device. A digital readout 30 provides data generated by software algorithms according to the present invention.

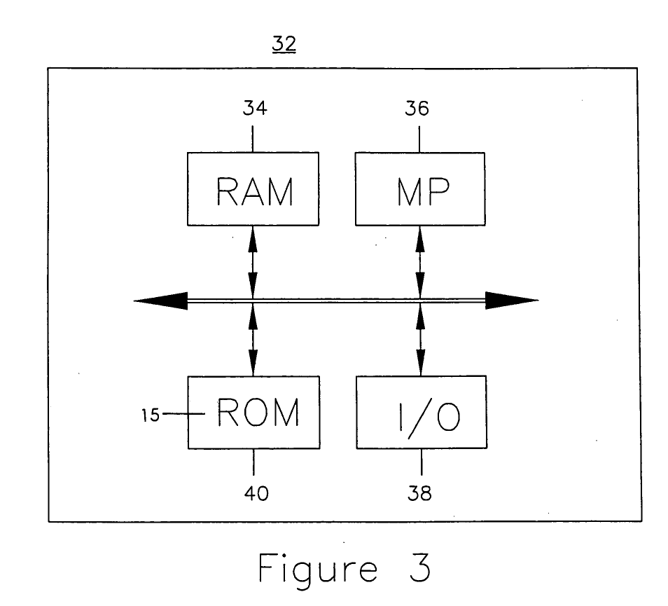

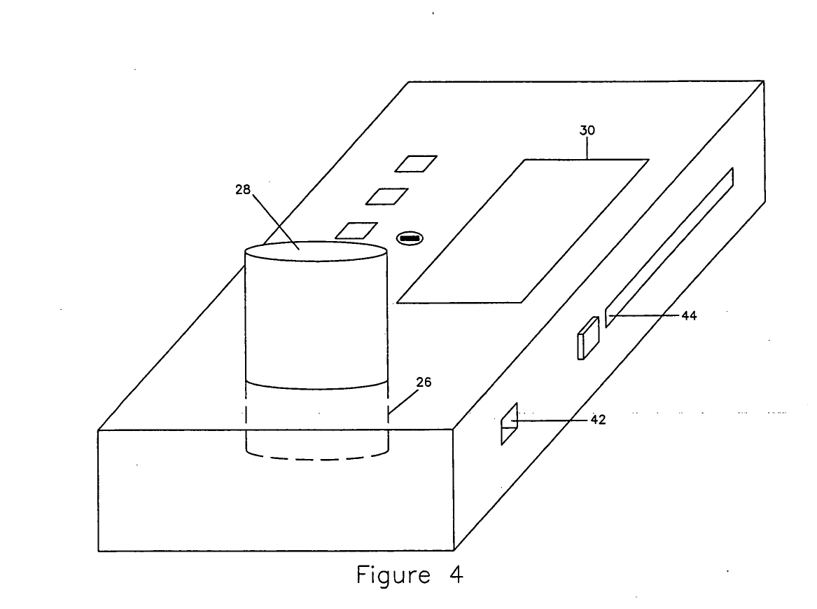

FIG. 3 is a program 15 stored in the ROM 40 that analyzes the blood sample and indicates a biological condition in conjunction with a microprocessor. This is displayed on the digital readout 30. As shown in FIG. 4, the output can be stored on a disk 44 or exported to a PC, POA or memory via port 42.

A number of empirical studies have produced relationships between blood characteristics and specific medical, dietary and health conditions. These are built into the system software. The invention can thus detect a series of dietary and medical conditions. Thrombocyte aggregation indicates small clots, narrowing blood vessels, impaired circulation, high cholesterol, candidiasis and headaches. Target Cells indicate that red blood cells have lost their capabilities to carry oxygen which will cause tiredness and lethargy. This may indicate low iron and insufficient bile. L Forms or Mesosomes indicate low immunity, frequent colds, high blood sugar, junk foods or embryonic bacteria.

Protein Linkage indicates that the body is having difficulty digesting protein and may indicate an amino acid deficiency. Red forms usually indicate that an infection is present. Fungal forms indicate that fungi are present. There are thirty different fungal forms that may be observed which are not necessarily Candida Albicans. Ovalocytes indicates an iron deficiency, depression, folic acid, B-12 deficiency, and an endocrine imbalance.

Parasitized RBC's indicates that bacteria are inside of the RBC and low immunity. Osmoplasts denote an osmotically fragile cell without spherical shape and retaining a rod form. Polkilecytosis indicates a deformed red blood cell that reveals an antioxidant deficiency and/or liver conditions.

Easimophil indicates allergies, parasites, dark circles under eyes, eczema, and edema. Echimocytes indicates deteriorating RBC's and possible kidney conditions. Mycoplasmas could result in arthritis, pneumonia, genitor-urinary tract and neurological diseases.

The invention can detect anisocytosis, with more than a normal variation in size, which can occur due to an iron deficiency, liver involvement and anemia. The invention can also be used to detect leukocytosis or increased white cell count or activity while leukopemia decreased white cell count or activity.

Rouleau indicates physical or mental stress, decreased amounts of oxygen and nutrients for the body to utilize, and fatigue and/or weakness. Erythrocyte Aggregation indicates a condition with saturated fat and abnormal protein causing the red blood cells to stick together and a possible enzyme or EFA deficiency.

Chylous indicates that the liver is having difficulty cleaning material of blood fats. This is best correlated with what was eaten prior to the test. The existence of plaque indicates poor circulation, calcium imbalance, cold hands and feet, and reduced energy or pain in the calves.

Spicules or fibria indicates toxicity of the liver and/or bowel. The existence of crystals indicates the inability of the body to absorb calcium. The crystals appear red, yellow or black due to bowel or liver toxicity, along with constipation or the existence of heavy metals. The appearance of macrocytes indicate a food allergy, poor absorption, B-12 deficiency and/or folic acid deficiency. Microcytes is an indicator of low iron.

The invention can also be used to detect transitionals, CWD forms for color micrographs, and atypical growth of either bacteria, yeast, toxicity or all. Neutrophilic Viability indicates poor immunity, infectious states, and malabsorption. Neutrophilic Hypersegmentation indicates poor immunity, depression, PMS, and deficiency. Candida indicates leaky gut, low immune response, parasitic activity, the detection of B-lymphocyters allergic or other immune responses. T-Lymphocytes indicate a cell mediated immunity. Acanthocytes suggests liver and spleen stress, vitamin E deficiency and hypercholesterolemia.

The invention can be used to indicate bacteria-potential infectious states; protoplasts—bacteria pool; parasites; worms; GTF; mutation and the level of normocytes healthy which are red cells able to transport oxygen and nutrients.

The present invention has been described with reference to the above discussed preferred embodiment. It is to be appreciated that the true nature and scope of the invention is to be determined with reference to the claims.

1. An apparatus for testing and determining a biological function based upon blood samples comprising:
a specimen holder for holding a blood sample;
a light on the specimen;
a molecular imager for viewing the specimens; and
a software program for determining a medical problem based upon an aspect of the blood sample specimen.

2. An apparatus for testing and determining a biological feature of a blood sample comprising:
a specimen holder for holding a blood sample;
a light for lighting the specimen of blood;
a molecular imager for imaging the specimen; and
a software program under the control of a microprocessor for determining a dietary problem based upon a feature of a blood sample specimen.

3. An apparatus for testing and determining a biological feature of a blood sample comprising:
a specimen holder for holding a blood sample;
a light for lighting the specimen of blood;
a molecular imager for imaging the specimen;
a software program under the control of a microprocessor for determining a dietary problem based upon a characteristic of the blood sample specimen; and
an output display for displaying the determined dietary problem.
